(19) United States
(12) Patent Application Publication
(10) Pub. No.: US 2025/0025019 A1
(43) Pub. Date: Jan. 23, 2025

(54) VIDEO LARYNGOSCOPE WITH AUTOMATIC ZOOM EFFECTS

(71) Applicant: Covidien LP, Mansfield, MA (US)

(72) Inventors: Peter D.C. Inglis, Boulder, CO (US); Derek S. Tata, Longmont, CO (US)

(73) Assignee: Covidien LP, Mansfield, MA (US)

(21) Appl. No.: 18/776,775

(22) Filed: Jul. 18, 2024

Related U.S. Application Data
(60) Provisional application No. 63/514,722, filed on Jul. 20, 2023.

(51) Int. Cl. A61B 1/00; A61B 1/267

(52) U.S. Cl. CPC A61B 1/000096; A61B 1/2673

(57) ABSTRACT

Disclosed is a video laryngoscope that automatically adjusts display of a portion of an acquired image. The video laryngoscope may detect patient anatomy and/or a tool present in an image captured by a camera of the video laryngoscope. Detection of patient anatomy and/or a tool may be performed via image recognition rules and/or machine learning (ML) models. Based at least on the detected tool(s), a portion of the acquired image may be selected for display. For example, a display region of an acquired image may be selected to include both detected patient anatomy and a portion of a detected tool. Display of the acquired images may be progressively adjusted as a detected tool moves toward or away from the patient anatomy, until a maximum or minimum display region is reached.

### CROSS-REFERENCE TO RELATED APPLICATIONS

This application claims the benefit of U.S. Provisional Application No. 63/514,722 filed Jul. 20, 2023, entitled “Video Laryngoscope with Automatic Zoom Effects,” which is incorporated herein by reference in its entirety.

### INTRODUCTION

Laryngoscopes are commonly used during intubation of a patient (e.g., an insertion of an endotracheal tube into a trachea of the patient). In video laryngoscopy, a medical professional (e.g., a doctor, therapist, nurse, clinician, or other practitioner) views a real-time video feed, captured via a camera of the video laryngoscope, of the patient's larynx on a display screen to facilitate navigation and insertion of tracheal tubes within the airway. A portion of the real-time video feed may be shown, based on a size of the display screen of the video laryngoscope.

It is with respect to this general technical environment that aspects of the present technology disclosed herein have been contemplated. Furthermore, although a general environment is discussed, it should be understood that the examples described herein should not be limited to the general environment identified herein.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

Among other things, aspects of the present disclosure include systems and methods for automatic zooming of an image from a camera of a video laryngoscope. In an aspect, a method for automatic cropping by a video laryngoscope is disclosed. The method includes acquiring a first image from a video feed of a camera of the video laryngoscope, the first image including a first portion of a tool and patient anatomy. The method also includes detecting the tool in the first image. Based on the detected tool in the first image, the method includes automatically selecting a first display region of the first image, the first display region including the patient anatomy and a tip of the tool. Additionally, the method includes acquiring a second image from the video feed, the second image including a second portion of the tool and the patient anatomy. The method includes detecting the tool in the second image. Based on the detected tool in the second image, the method includes automatically selecting a second display region of the second image, the second display region including the patient anatomy and the tip of the tool.

In an example, the patient anatomy is enlarged in the second display region relative to the first display region. In another example, the method further includes displaying the first display region and the second display region in real time at a display of the video laryngoscope. In a further example, the tip of the tool in the first display region and the second display region has a same height when the first display region and the second display region are displayed, and the same height is a distance from an end of the tip of the tool to a bottom edge of one of the first display region or the second display region. In yet another example, the patient anatomy includes vocal cords and the tool is an endotracheal tube. In still a further example, the tip of the tool is a distal end of the endotracheal tube positioned distally from a cuff of the endotracheal tube.

In another aspect, a method for automatic cropping by a video laryngoscope is disclosed. The method includes acquiring an image using a camera of the video laryngoscope, the image including a portion of a tool. The method also includes providing at least a portion of the acquired image as input into a trained machine learning (ML) model. The method further includes receiving detection of the tool as output from the trained ML model. Based on the tool detection, the method includes zooming out to a portion of the acquired image including patient anatomy and a tool portion of the tool having a tool height. Additionally, the method includes displaying the zoomed-out portion of the acquired image at a display of the video laryngoscope.

In an example, the tool height is less than 20 mm when the zoomed-out portion of the acquired image is displayed at the display of the video laryngoscope. In another example, displaying the zoomed-out portion of the acquired image includes fitting the zoomed-out portion to the display. In a further example, the aspect ratio of the zoomed-out portion and the display are the same. In yet another example, the method further includes detecting a progression of the tool towards the patient anatomy; and progressively zooming in to portions of images acquired by the camera as the tool progresses towards the patient anatomy. In still a further example, the zoomed-in portions include the tool portion having the tool height.

In another aspect, a video laryngoscope is disclosed. The video laryngoscope includes a handle portion; a display screen coupled to the handle portion; camera, positioned at a distal end of the blade portion, that acquires a video feed while the video laryngoscope is powered on; a memory; and a processor. The processor operates to acquire a first image from a video feed of a camera of the video laryngoscope, the first image including patient anatomy. The processor further operates to display a first portion of the first image on the display screen and acquire a second image from the video feed, the second image including a tool and the patient anatomy. The processor also operates to detect the tool in the second image. Based on detecting the tool in the second image, the processor operates to display a second portion of the second image, wherein the second portion is larger than the first portion thereby providing a zoom-out effect.

In an example, first portion is cropped from the first image, wherein the second portion is cropped from the second image, and wherein the second portion includes a tip of the tool and the patient anatomy. In another example, the processor further operates to: acquire a third image from the video feed, the third image including the patient anatomy and the tool after the tool has been further distally inserted towards the patient anatomy; detect the tool in the third image; and based on detecting the tool in the third image, display a third portion of the third image, wherein the third portion is smaller than the second portion thereby providing a zoom-in effect as compared to the displayed second portion of the second image.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the description which follows and, in part, will be apparent from the description, or may be learned by practice of the disclosure.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a schematic of an example patient environment including a video laryngoscope.

FIG. 2 is a perspective view of the video laryngoscope of FIG. 1.

FIG. 3 is a block diagram of components of the video laryngoscope.

FIGS. 4A-4D show example display images of an acquired image captured from a camera of a video laryngoscope.

FIG. 5 shows an example flow of display screens of a video laryngoscope with automatic zoom.

FIGS. 6 and 7 show example methods for automatic zoom with a video laryngoscope.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

Video laryngoscopes are commonly used during intubation of a patient (e.g., an insertion of an endotracheal tube into a trachea of the patient). During intubation, the patient's airway and larynx may be visualized by a medical professional (e.g., a doctor, therapist, nurse, clinician, or other practitioner), such as via video laryngoscopy. In video laryngoscopy, the medical professional may view a real-time video feed of the patient's larynx, other patient anatomy, or other objects or structures in the upper airway of the patient, as captured via a camera of the video laryngoscope and displayed on a display screen of the video laryngoscope. The video feed may assist a medical professional to visualize the patient's airway and facilitate manipulation and insertion of a tracheal tube. A portion of the real-time video feed may be shown, based on a size of the display screen of the video laryngoscope.

The acquired camera images from the real-time video feed may be larger than, and/or have different aspect ratios than, a display screen of the video laryngoscope. The image displayed at the display screen (e.g., a display image) may thus include some, but not all, of an acquired image (e.g., a portion of an acquired image is displayed at the display screen as a display image). With different sized screens, different regions and/or portions of the acquired image is displayed. For example, images displayed at larger screens may include more of the acquired image than images displayed at smaller screens (e.g., more of the posterior view is shown). Display of a larger region of the acquired images may cause certain patient anatomy (e.g., vocal cords, larynx) to appear small or off-center at a top portion of the screen. This smaller and off-center viewing of patient anatomy at the display screen may cause clinicians to think, based on the displayed image, that there is an issue with the patient's anatomy (e.g., the patient anatomy is anterior and/or small). In some instances, however, a posterior view of the patient (e.g., more of the acquired image shown) may assist a clinician to see an inserted tool (e.g., for steering and placement of the tool) and to reduce a likelihood of soft palate injury during tool movement. After the tool passes the posterior region, however, the posterior view may not be as relevant or useful for the clinician. Instead, after a tool has passed the posterior region, adjusting display of the acquired image to cause a zooming effect onto certain patient anatomy (e.g., the vocal cords) may be more desirable for a clinician (e.g., as a clinician targets the vocal cords during intubation).

Provided herein are systems and methods for automatically adjusting display of an image acquired by a camera of a video laryngoscope. As used herein, zooming causes a portion of the acquired image to be enlarged or reduced on the display screen. For instance, zooming may include selecting a portion or region of an acquired image, for display, and fitting the portion of the acquired image to a display of the video laryngoscope. For example, zooming in may include enlarging a portion of an acquired image at a display of the video laryngoscope (e.g., via selecting a region of the acquired image and fitting/filling the region to the display, resizing the image). As another example, zooming out may include shrinking or reducing a portion of an acquired image at a display of the video laryngoscope (e.g., via selecting a region of the acquired image and fitting/filling the region to the display, resizing the image). Selection of a display region may include cropping of the acquired image to the display region (e.g., a crop region). As used herein, cropping may include selecting a region of the acquired image. Cropping the acquired image is for display of the crop region and may not cause loss of image data outside of the crop region. An acquired image may be cropped to a crop region and fitted to a display to cause a zoom effect at the display. The video laryngoscope may be capable of detecting patient anatomy and/or tool(s) present in an image captured by a camera of the video laryngoscope. Detection of patient anatomy and/or tool(s) may be performed via image recognition rules and/or machine learning (ML) models. Based on the detected patient anatomy and/or tool(s) detected, a display of the acquired image may be adjusted (e.g., resized, such as by selecting a region or cropping to a crop region and filling/fitting the region to a display). For example, if patient anatomy is detected and no tool is detected, the acquired image may be adjusted to include display of the patient's vocal cords (e.g., selecting a region or cropping to cause a zooming effect about patient anatomy). If the patient anatomy and at least one tool is detected, the acquired image may be adjusted to include display of the patient anatomy and a portion of the tool(s). The displayed portion/region of the acquired image may be resized to fit/fill the display screen (e.g., zooming-in/enlarging about the patient anatomy and the portion of the tool or zooming-out/shrinking about the patient anatomy and the portion of the tool).

The displayed portion/region of the acquired images may be progressively resized as the tool(s) move toward the patient anatomy (e.g., moving distally in the patient) so that the patient anatomy is progressively enlarged and expanded to fill more of the display screen. This progressive adjustment/resizing may continue until a minimum display region is reached (e.g., a limit on how large the patient anatomy appears on the display screen). As the detected tool is retracted from the patient, the acquired image may also be progressively resized in a reverse manner. For example, as the tool moves away from the patient anatomy, the progressive resizing may cause a zooming-out effect of the patient anatomy being progressively shrunk and filling less of the display screen. This may continue until a maximum display region is reached (e.g., a limit on how small the patient anatomy appears on the display screen). As new tool(s) are detected, the display portion of the acquired images may be adjusted accordingly. Adjustment of the display portion (e.g., zooming or cropping and filling/fitting) of the acquired images may be automatic.

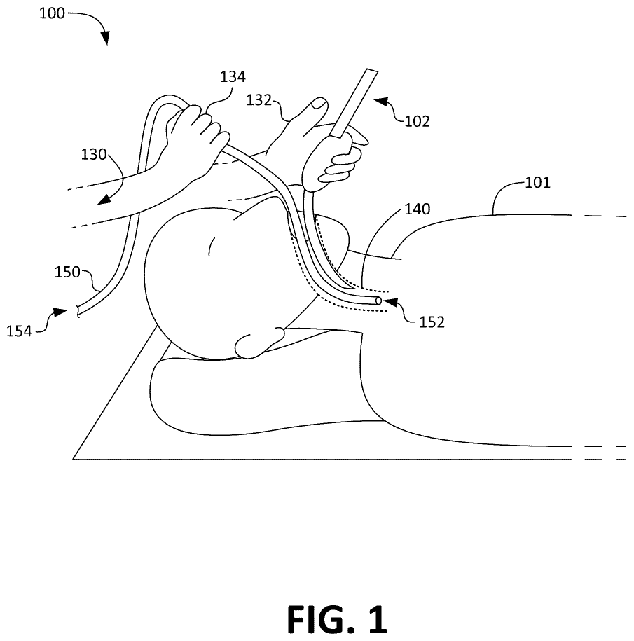

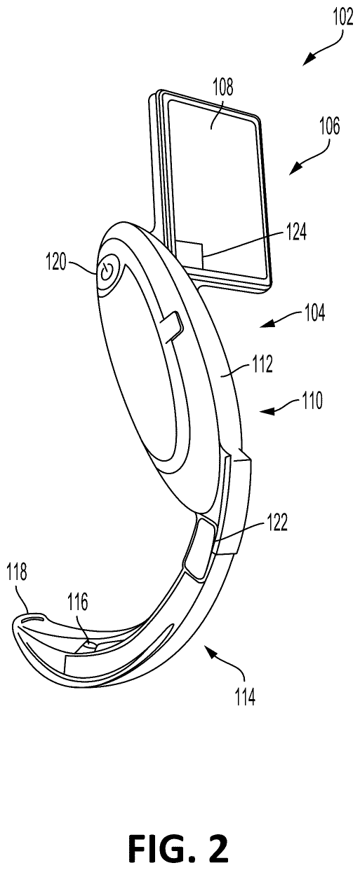

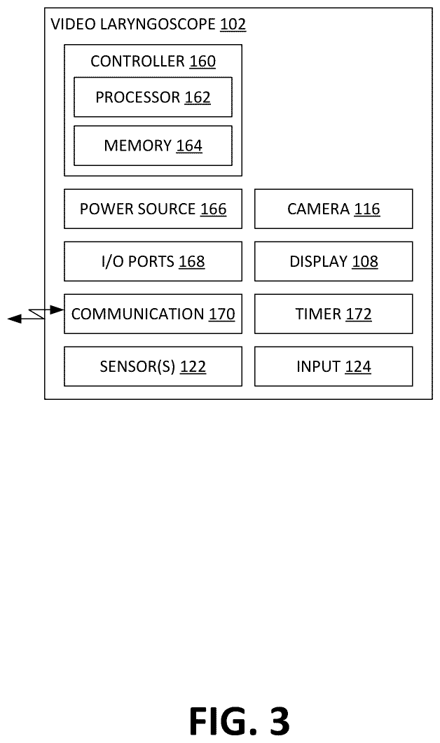

FIG. 1 shows an example patient environment 100 including a video laryngoscope 102. The patient environment 100 may be any room where an intubation is being performed, such as a medical suite in a hospital or other care setting, an operating or other procedure room, patient recovery room, an emergency intubation setting, or other environments. As described herein, the video laryngoscope 102 may be used for visualizing an airway of a patient 101 and/or a tool 150 (e.g., introducer, endotracheal tube, etc.) inserted into the airway of the patient 101, based on an image from a camera 116 of the video laryngoscope 102. The video laryngoscope 102 may be positioned in a patient's airway 140 concurrently with one or more tools 150, such as an endotracheal tube, an introducer (e.g., to place the endotracheal tube), a bougie, forceps, flexible scope, and/or any other tool for facilitating intubation of a patient 101. Aspects of the video laryngoscope 102 are further shown in FIGS. 2 and 3.

In FIG. 1, a medical professional 130 is shown holding a video laryngoscope 102 in a first hand 132 (e.g., a left hand 132 of the medical professional 130) and a tool 150 in a second hand 134 (e.g., a right hand 134 of the medical professional 130). As further described herein, the video laryngoscope 102 may be positioned in the airway 140 of the patient 101 to manipulate and/or visualize the patient's airway 140, such as with an arm 114 or blade 118. Visualization of the airway 140 of the patient 101 may include viewing patient's anatomy (e.g., larynx, trachea, esophagus, vocal cords, etc.) with a camera 116 of the video laryngoscope 102. The medical professional 130 may move the tool 150 proximally (e.g., retract the tool 150) or distally (e.g., advance the tool 150), while watching the resulting images from the camera 116 of the video laryngoscope 102 on the display 108 of the video laryngoscope 102.

The images acquired from the camera 116 of the video laryngoscope 102 may thus include patient anatomy and/or a portion of a tool 150 inserted into the airway and visible by the camera 116. In some examples, the acquired images may not include a tool 150, such as when a tool 150 is not positioned in the airway of the patient 101 (e.g., prior to insertion and after removal/retraction). In other examples, the distance between a portion of the tool 150 in the image and the patient anatomy in the image may vary, based on the relative position of the tool 150 and the patient anatomy in the airway.

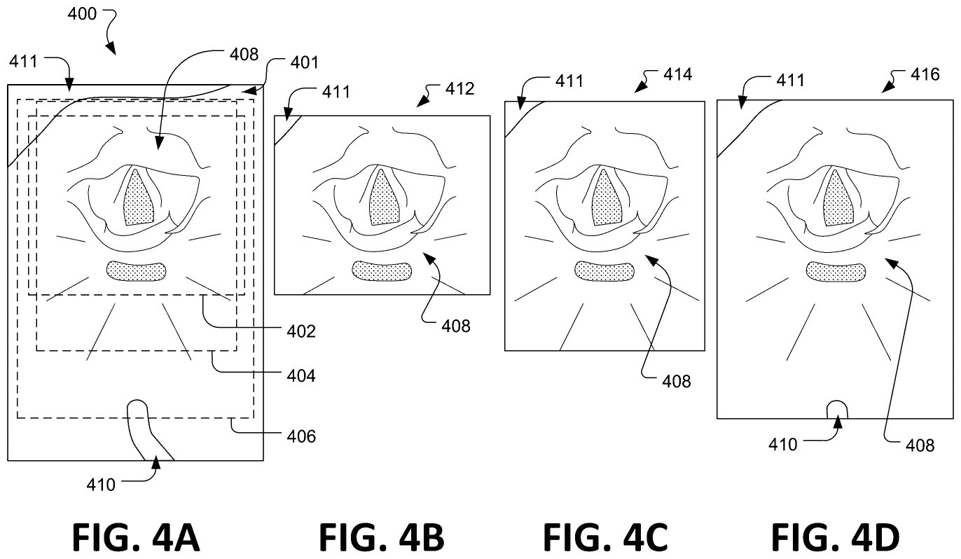

The images acquired by the camera 116 may be larger than the images displayed at the display 108 of the video laryngoscope 102 (e.g., a portion of the acquired image may not be displayed). In some examples, display of a display portion of the acquired images may be adjusted based on a size and/or shape of the display 108. For example, smaller displays 108 may include less visual information from an acquired image (e.g., a display region is smaller or more of the acquired image is not in the display region or cropped out) and larger displays 108 may include more visual information from an acquired image (e.g., a display region is larger or less of the acquired image is not in the display region or cropped out). An example of how a display region may be adjusted differently for different displays 108 is further discussed with respect to FIGS. 4A-4D.

Display of an acquired image may be automatically adjusted at a display 108 of a video laryngoscope 102. As described herein, adjusting display of an acquired image means to display an inside of a display region and not display portions of the acquired image outside of the display region. The display region, or the display image, is then displayed at a display 108 of the video laryngoscope 102. The display region may thus have the same aspect ratio as the display 108. Displaying the display image may include enlarging of the display image to fill the display 108 (e.g., a zoom-in effect) or shrinking of the display image to fill the display 108 (e.g., a zoom-out effect). In some instances, the selected display region may be sized for display without a zoom effect. Selection of the display region (e.g., crop region) of an acquired image may be performed after the image has been acquired and analyzed by the video laryngoscope (e.g., for detection of a tool and/or patient anatomy).

Images acquired by the camera 116 of the video laryngoscope 102 may be analyzed to detect patient anatomy and/or tool(s) in the images. Based on detection of patient anatomy and/or tool(s), a display region of the acquired images may be selected for display. If no tools are detected in an image, the display region may be selected based on patient anatomy and/or display size/shape. In an example, patient anatomy is detected and centered in a display region of the display image. Alternatively, the video laryngoscope may not analyze an image for detection of patient anatomy (e.g., only tool detection) and a preselected display region (e.g., based on display size/shape) may be displayed regardless of patient anatomy.

If one or more tools are detected, the display region may be selected or adjusted to include both the patient anatomy and a portion of the tool to be displayed at a display 108 of the video laryngoscope 102. Displaying both the patient anatomy and a portion of a tool inserted into the airway may assist an operator in placement and/or movement of the tool. When a tool is not detected, patient anatomy may be zoomed and/or centered in the display image to reduce an amount of patient anatomy that may be less helpful during intubation (e.g., portions of the hypopharynx, epiglottis, oropharynx, etc.). The available display regions may be limited or restricted to set of sizes and/or configurations. For example, there may be a minimum display region (e.g., maximum zoom-in effect) and/or a maximum display region (e.g., maximum zoom-out effect). For instance, a maximum display region may be based on a width and/or height of the display region being the same as the acquired image (e.g., all of the width and/or height of the acquired image is included in the display region). In another instance, a minimum display region may be based on distance between a detected tool and the patient anatomy, distance between the tool and a top border of the selected display region and/or acquired image, a maximum fill of the patient anatomy, and/or image quality considerations.

Tool detection and/or patient anatomy detection by the video laryngoscope 102 may be based on a single image captured by the camera 116 of the video laryngoscope 102. The image may be a real-time, still-shot frame from a real-time video feed of a camera, such as a camera 116 of a video laryngoscope 102. Recognition or detection of the tool 150 from the single frame may be based on image recognition rules (e.g., coded heuristics or rule-based algorithms) or artificial intelligence (AI) algorithms and/or machine learning (ML) models (e.g., trained). The single frame may be the only input into image recognition rules or algorithms/model. In other examples, multiple images from the video feed may be used for detection of anatomy and/or tools. If using a trained ML model, the model may be a neural network, such as a deep-learning neural network or convolutional neural network, among other types of AI or ML models. Other types of models, such as regression models, may also or alternatively be used. Training of the model may be based on one or more still-shot images associated with different tools. The trained model may receive and detect patient anatomy and/or tool(s) in the airway, trained based on comparisons or analysis of the sets of training images.

Tool detection and/or selection of a display region of an acquired image can be performed on the video laryngoscope 102 itself and in real time (e.g., low latency). Because tool detection and/or display region selection is based on image analysis, any tool that is inserted into the patient (and within view of the camera of the video laryngoscope) may be detectable. Additionally, no user input is required for tool detection and/or display region selection. For instance, in some examples, tool detection and/or selection of the display region and/or fitting/filling a selected display region to a display is performed automatically by the video laryngoscope 102.

Image analysis for tool detection may persist in a continuous loop. In a continuous loop analysis, contemporaneous image frames may be analyzed in real time. For example, each image frame of a video feed (e.g., frames acquired at 30 frame per second) may be analyzed. Thus, a display region of each image frame of the video feed may be selected according to the present technology. In examples wherein not all frames of a video feed are displayed at the display 108 of the video laryngoscope 102, a subset of the total image frames of a video feed (e.g., the subset of frames displayed to an operator) may be analyzed. Alternatively, images may be analyzed at different intervals depending on when a tool is not detected in the images. For example, prior to tool detection and/or after tool removal/retraction, a subset of the total image frames of the video feed may be analyzed. Alternatively, when a tool is detected each of the image frames of the video feed may be analyzed. A subset of the total image frames (e.g., as may be analyzed when a tool is not detected) may be every second, third, fourth, etc. frame. Alternatively, image frames (e.g., prior to tool detection and after tool removal/retraction) may be analyzed in preset intervals (e.g., every 0.1 seconds, every 0.2 seconds, etc.) as may be tracked by a timer of the video laryngoscope 102.

FIG. 2 shows a perspective view of the video laryngoscope 102. As shown, the video laryngoscope 102 has a body 104 (e.g., reusable body). The body 104 includes a display portion 106 having a display screen 108 that is configured to display images and/or other data, a handle portion 110 having a handle 112 that is configured to be gripped by the medical professional during the laryngoscopy procedure, and an elongate portion or arm 114 that supports a camera 116 and light source (e.g., light-emitting diodes (LEDs)) that is configured to obtain images, which may be still-shot images and/or moving images (e.g., a video feed). The camera 116 and light source may be incorporated on the distal end of the arm 114. The light source may be provided as part of the camera 116 or separate from the camera 116 on the blade 118 or arm 114.

In examples, the display portion 106 and the handle portion 110 may not be distinct portions, such that the display screen 108 is integrated into the handle portion 110. In the illustrated embodiment, an activating cover, such as a removable laryngoscope blade 118 (e.g., activating blade, disposable cover, sleeve, or blade), is positioned about the arm 114 of the body 104 of the laryngoscope 102. Together, the arm 114 of the body 104 and the blade 118 form an insertable assembly that is configured to be inserted into the patient's oral cavity. It should be appreciated that the display portion 106, the handle portion 110, and/or the arm 114 that form the body 104 of the laryngoscope 102 may be fixed to one another or integrally formed with one another (e.g., not intended to be separated by the medical professional during routine use) or may be removably coupled to one another (e.g., intended to be separated by the medical professional during routine use) to facilitate storage, use, inspection, maintenance, repair, cleaning, replacement, or interchangeable parts (e.g., use of different arms or extensions with one handle portion 110), for example.

The handle 112 and/or arm 114 may include one or more sensors 122 capable of monitoring functions (e.g., different, additional, and/or advanced monitoring functions). The sensors 122 may include a torque sensor, force sensor, strain gauge, accelerometer, gyroscope, magnet, magnetometer, proximity sensor, reed switch, Hall effect sensor, etc. disposed within or coupled to any suitable location of the body 104. The sensors 122 may detect interaction of the video laryngoscope 102 with other objects, such as a tool 150, physiological structures of the patient (e.g., teeth, tissue, muscle, etc.), or proximity of a tube, introducer, bougie, forceps, scope, or other tool.

The laryngoscope 102 may also include a power button 120 that enables a medical professional to power the laryngoscope 102 off and on. The power button 120 may also be used as an input device to access settings of the video laryngoscope 102. Additionally, the video laryngoscope 102 may include an input button, such as a touch or proximity sensor 124 (e.g., capacitive sensor, proximity sensor, or the like) that is configured to detect a touch or object (e.g., a finger or stylus). The touch sensor 124 may enable the medical professional operating the video laryngoscope 102 to efficiently provide inputs or commands, such as inputs to indicate insertion of a tool 150 into the patient's airway, inputs that cause the camera 116 to obtain or store an image on a memory of the laryngoscope, and/or any other inputs relating to function of the video laryngoscope 102.

FIG. 3 is a block diagram of components of the video laryngoscope 102. As shown, the video laryngoscope 102 may include various components that enable the video laryngoscope 102 to carry out the techniques disclosed herein. For example, the video laryngoscope 102 may include the display screen 108, the camera 116, a light source (e.g., which may integrated into the camera or separate from the camera), sensor(s) 122, and input (e.g., touch sensor) 124, as well as a controller 160 (e.g., electronic controller), one or more processors 162, a hardware memory 164, a power source (e.g., battery) 160, input/output (I/O) ports 168, a communication device 170, and a timer 172. In some embodiments, the timer 172 may track relative time (e.g., a start time, an end time, a frequency of image frame sampling), which may be referenced to acquire still-shot input images for analysis, detect the presence of a tool in an image, and/or adjust display of an acquired image (e.g., via selection of a display region and/or cropping and/or filling/fitting of a portion of the acquired image for display).

The communication device 170 may enable wired or wireless communication. The communication devices 170 of the video laryngoscope 102 may communicatively couple with communication devices of a remote device (e.g., a care-facility computer, remote viewing device, etc.) to allow communication between the video laryngoscope 102 and the remote device. Wireless communication may include transceivers, adaptors, and/or wireless hubs that are configured to establish and/or facilitate wireless communication with one another. By way of example, the communication device 170 may be configured to communicate using the IEEE 802.15.4 standard, and may communicate, for example, using ZigBee, WirelessHART, or MiWi protocols. Additionally or alternatively, the communication device 170 may be configured to communicate using the Bluetooth standard or one or more of the IEEE 802.11 standards.

In some examples, the video laryngoscope 102 includes electrical circuitry configured to process signals, such as signals generated by the camera 116 or light source, signals generated by the sensor(s) 122, and/or control signals provided via inputs 124 or automatically. The processors 162 may be used to execute software. For example, the processor 162 of the video laryngoscope 102 may be configured to receive signals from the camera 116 and execute software to acquire an image, analyze an image, detect a tool and/or patient anatomy, select a display region of the acquired image for display, display the display region (e.g., which may include resizing of the to fill/fit a display), etc.

The processor 162 may include multiple microprocessors, one or more “general-purpose” microprocessors, one or more special-purpose microprocessors, and/or one or more application specific integrated circuits (ASICS), or some combination thereof. For example, the processor 162 may include one or more reduced instruction set (RISC) processors.

The hardware memory 164 may include a volatile memory, such as random access memory (RAM), and/or a nonvolatile memory, such as read-only memory (ROM). It should be appreciated that the hardware memory 164 may include flash memory, a hard drive, or any other suitable optical, magnetic, or solid-state storage medium, other hardware memory, or a combination thereof. The memory 164 may store a variety of information and may be used for various purposes. For example, the memory 164 may store processor-executable instructions (e.g., firmware or software) for the processor 162 to execute, such as instructions for processing signals generated by the camera 116 to generate the image, provide the image on the display screen 108, analyze an image via a trained model, detect a tool and/or patient anatomy in an image, select a display region and/or crop the image, adjust (e.g., shrink/reduce or enlarge) the image for display, etc. The hardware memory 164 may store data (e.g., acquired images, training images, image recognition rules, AI or ML algorithms, trained models, etc.), instructions (e.g., software or firmware for generating images, storing the images, analyzing the images, adjusting the images for display, etc.), and any other suitable data.

FIGS. 4A-4D show example display images 412-416 selected from a display region 402-406 of an acquired image 400 captured from a camera of a video laryngoscope. In particular, FIGS. 4B-4D show that different portions (e.g., different display regions) of an acquired image 400 may be displayed for different display sizes. The acquired image 400 may include patient anatomy 408, a portion of a tool 410, and a portion of a blade 411. In some examples, the acquired image 400 may be larger than, or have a different aspect ratio than, a display screen of the video laryngoscope for display of the display images 412-416. In such examples, the acquired image 400 may be cropped to a display region 402-406, based on the size and shape (e.g., aspect ratio) of the display screen (e.g., display regions 402-406 associated with display images 412-416).

In the examples shown in FIGS. 4B-4D, a portion of the acquired image 400 is selected (e.g., a display region 402-406) and the display region is displayed as an associated display image 412-416. In this instance, the display region is not modified prior to display (e.g., the display region is pre-fitted for display characteristics such that no shrinking/reducing or enlarging of the display image is performed and no zoom effect is realized). The display images 412-416 may have different aspect ratios and/or dimensions. For example, the display image 412 shown in FIG. 4B may be associated with a first display region 402 with a first height and a first width. Similarly, the display image 414 shown in FIG. 4C may be associated with a second display region 404 with a second height and a second width, and the display image 416 shown in FIG. 4D may be associated with a third display region 404 with a third height and a third width. The display regions 402-406 may be selected based on a predetermined offset 401 or spacing 401 from the top edge of the acquired image 400. An offset 401 or spacing 401 may be predetermined based on expected camera obfuscations or image quality considerations, such as obscuring of the screen by a portion of the blade 411 or different light reflections/refractions near an edge of the acquired image 400.

As dimensions of the display regions 402-406 differ, different portions of the acquired image 400 are displayed. For example, the third display image 416 may show more of the posterior view (e.g., show more anatomy) than the first display image 412 or the second display image 414. This may be, in part, because the height of the third display region 406 is larger than the height of the first display region 402 and second display region 404. Including more of the posterior view in the display image 416 may cause the patient anatomy 408 to appear off-center towards the top of the screen and/or the patient anatomy 408 may have an illusion of being small because the patient anatomy 408 fills less of the display image 416.

Although the display images 412-416 shown in FIGS. 4B-4D do not show a zoom effect as compared with the display regions 402-406 shown in FIG. 4A, the display regions may be fitted or filled (e.g., resized) to a display such that a zoom effect is caused between the display image and the acquired image 400. For example, as further described herein, a display region 402-406 may have a fixed aspect ratio and may be made larger or smaller (e.g., including more or less of the acquired image 400), with the resulting display image 412-416 fitting or filling the display region 402-406 to a display screen (having a constant size and shape) of a video laryngoscope.

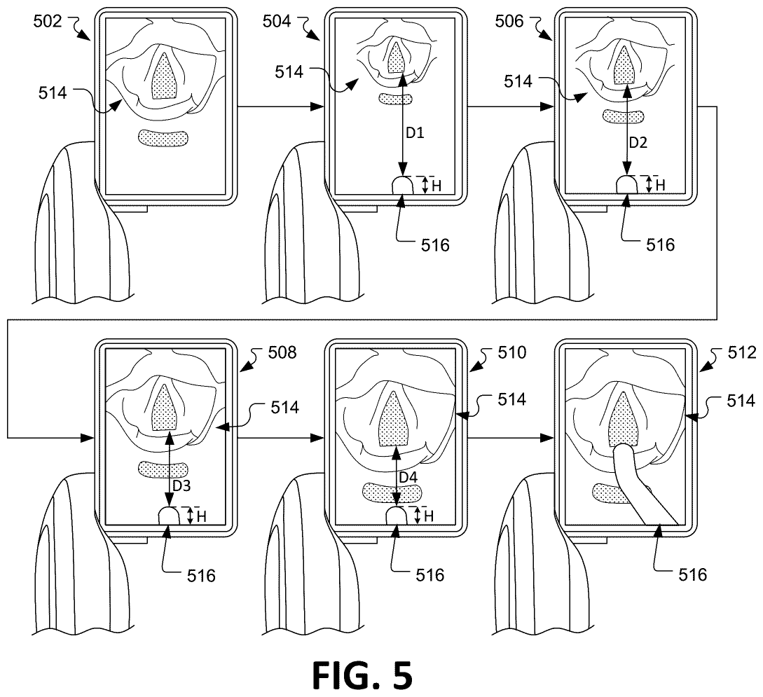

FIG. 5 shows an example flow of displays 502-512 of a video laryngoscope with automatic display region selection of an acquired image for display. The flow of displays 502-512 shows displaying of display regions of acquired images over time, based on detection of patient anatomy 514 and/or detection of a tool 516. At display 502, patient anatomy 514 is displayed by the video laryngoscope, without any portion of a tool. The display region of the acquired image, associated with the display, may be based on detection of the patient anatomy 514. For example, patient anatomy may be detected and a display region may be selected to enlarge and center the patient anatomy for display. Alternatively, when a tool is not detected, a display region may be selected based on predetermined sizing and positioning of the display region, without detection of patient anatomy. A predetermined display region may be associated with an initial zoom effect, which may not be a maximum zoom effect (e.g., may not be a minimum display region). The display 502 may show a display image that does not have a zoom effect (e.g., the display region was not modified, such as by filling or fitting, for display), such as the display regions described in FIGS. 4A-4D, above. Alternatively, the display image at display 502 may include a zoom effect relative to the acquired image, such that the patient anatomy 514 fills more of the display 502 and appears more centered at the display 502.

At display screen 504, a tool 516 is detected in the acquired image. The display image is selected from the acquired image (e.g., as a display region) to include a portion of the detected tool 516 and the patient anatomy 514. The portion of the tool included in the display image, when not at a maximum or minimum display region, may be determined based on a predetermined tool portion height H (e.g., 2 mm, 3 mm, 5 mm, 7 mm, 10 mm, 15 mm, 20 mm, etc.) and/or a tool component (e.g., at or above/distal to a cuff of an endotracheal tube), such that the bottom edge of the display region is adjusted to show a desirable amount of the tool. The predetermined tool portion height H may include a tip of the tool. The tool portion height H may be measured as a distance between a bottom edge of the display region and a distal end of the tool. The top edge of the display region may be based on inclusion of patient anatomy, removal of camera obfuscations, and/or an offset/spacing from the top edge of the acquired image. Display 504 may be a maximum display region (e.g., maximum zoom-out effect of the acquired image).

At displays 506-508, a display region of the acquired image is progressively adjusted based on tool detection. In the example depicted, as the tool moves (e.g., a distance D1-D4 is changed between the tool 516 and the patient anatomy 514 and/or top edge of the display region), the display region is changed to include a substantially constant tool portion height H and a constant top edge border, and the display region is fitted/filled to the display, accordingly. For example, a display region of acquired images are progressively adjusted, the tool portion height H, or tip of the tool, may remain unchanged and a distance between the tool 516 and the patient anatomy 514 (or a distance between the tool 516 and the top edge of the display region/display) may decrease. Thus, progressive adjustment of the display region and fitting/filling may result in change a size of the patient anatomy 514 relative to the display and may result in the illusion that the patient anatomy 514 changing size or distance from the camera (e.g., an appearance that the patient anatomy is enlarged or closer to the camera as a tool is inserted distally and an appearance that the patient anatomy is shrunk or further from the camera as the tool is removed/retracted from the patient).

At displays 510, 512, a minimum display region (e.g., maximum zoom effect) is reached. The minimum display region shown in displays 510, 512 may be different than an initial display region (e.g., when no tool is detected), such as shown at display 502. When a minimum display region is reached, the minimum display region is maintained, regardless of tool portion height H (e.g., a larger portion of a tool may be shown), until the tool is removed/retracted past the tool portion height H (e.g., a distance between the tool and the patient anatomy and/or top edge of the display region justifies increasing the display region). As the tool is removed/retracted from the patient, the display region may be changed to include a constant tool portion height H and a constant top edge border. For example, as the tool is removed/retracted, displays 502-510 may flow backwards (e.g., display 510 to display 508 to display 506 to display 504) until the tool is no longer detected and an initial display region is re-displayed (e.g., as shown in display 502). In some examples, multiple tools may be detected concurrently in the airway. In such an instance, the display region may be based on inclusion of at least a portion of each detected tool.

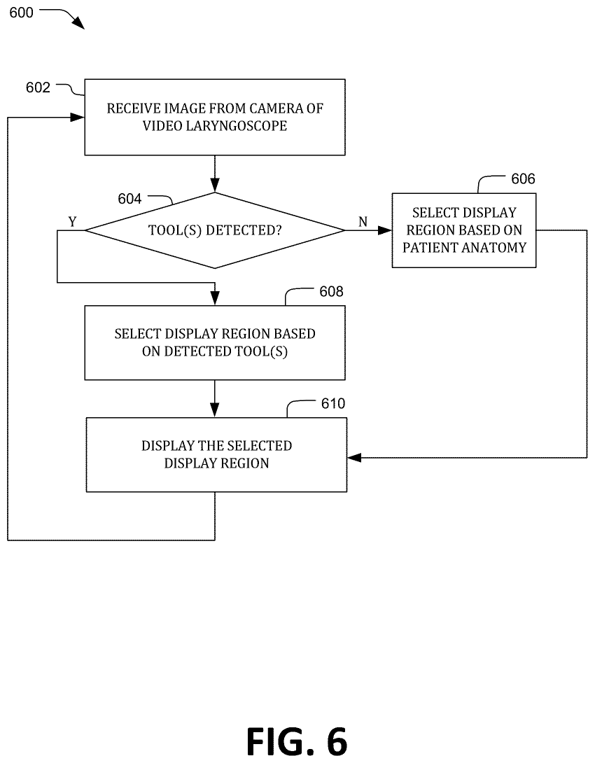

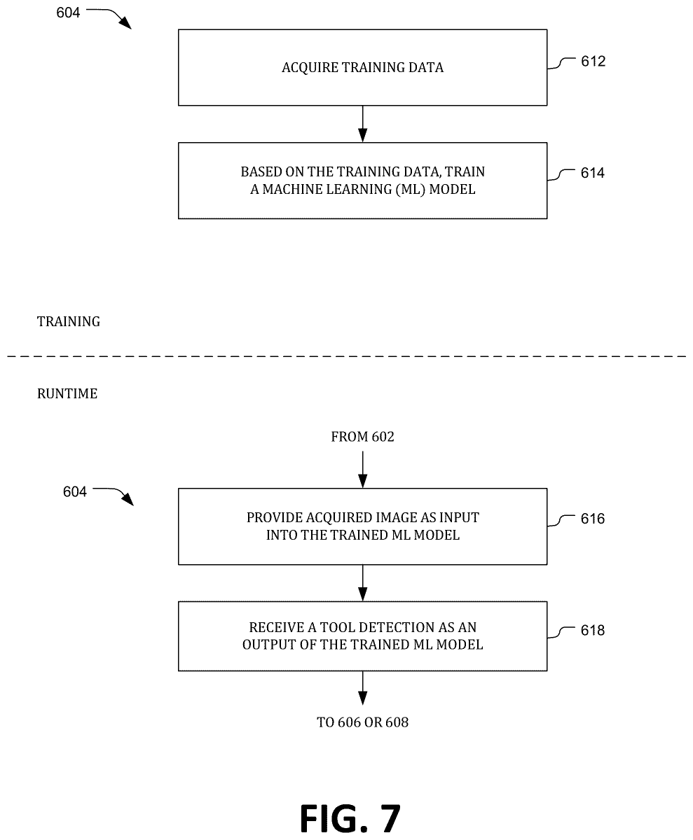

FIGS. 6-7 show example methods according to the disclosed technology. The example methods include operations that may be implemented or performed by the systems and devices disclosed herein. For example, the video laryngoscope 102, depicted in at least FIGS. 1, 2, and 3, may perform the operations described in the methods. In addition, instructions for performing the operations of the methods disclosed herein may be stored in a memory of the video laryngoscope (e.g., system memory 164 described in FIG. 3).

FIGS. 6-7 show example methods 600, 700 for automatic adjustment of display of a portion of an acquired image. Beginning with FIG. 6, at operation 602, an image is received at a camera of a video laryngoscope. The acquired image may be a raw/full-sized image from the camera. One or more of the dimensions of the acquired image may be larger than the dimensions of a display screen of the video laryngoscope. The acquired image may include a patient anatomy and/or a portion of a tool in the patient's airway.

At determination 604, tool detection is determined. In addition to tool detection, patient anatomy may be detected. The patient anatomy and/or tool(s) may be detected based on the acquired image. Detection of tool(s) and/or patient anatomy may be automatic and/or in real time. The detection of the tool(s) and/or patient anatomy may be determined by image recognition rules, AI algorithms, and/or ML models. In some examples, patient anatomy may not be detected. In such examples, a known or predetermined top edge border may be set based on an offset or spacing from a top edge of the acquired image.

Operation 604 is further described with respect to FIG. 7. As shown, the tool detection may be determined by image recognition rules, AI algorithms, and/or ML models. The machine learning (ML) model may be trained (e.g., as described in operations 612, 614) prior to runtime operations (e.g., operations 616, 618).

During training of the ML model, operations 612, 614 may be performed. At operation 612, training data is received. Training of the trained model may occur prior to the trained model's deployment/installation on the video laryngoscope. The training data may include a large set or sets of images that are labeled with respective corresponding classifications to train a foreign object detection algorithm. The training data may be labeled with the corresponding classes via manual classifications or through other methods of labeling images. Classifications for tool detection may include different tools and no tool. For example, multiple training images may be provided and labelled for no tool, a first tool, a second tool, a third tool, etc. (e.g., no tool in the image other than components of the laryngoscope such as a blade, a variety of endotracheal tubes, introducers or bougies, scopes, forceps, etc.). Tool(s) may be detected based on size, shape, shading, and/or relative positioning between two or more images (e.g., detected movement over time). In examples where relative positioning is used, the training data may include groupings of images over time and one or more image inputs may be received as input into the ML model during runtime.

In some examples, the training data images may be a portion of raw/full-sized images acquired by a camera of a video laryngoscope. For example, training images may be cropped images from a camera of a video laryngoscope, with the crop region including a portion of the acquired images in which a tool would most likely be visible. For instance, a crop region for the training data images may be a lower half, lower third, lower fourth of the acquired image, etc. Limiting the training data to relevant tool detection regions may remove noise from the training data.

At operation 614, the ML model is trained, based on the training data. Training the ML model with the training data set may include use of a supervised or semi-supervised training method or algorithm that utilizes the classified images in the training data. Once the trained model is generated, the trained model may be used to detect a tool in real time.

After the ML model is trained, the trained ML model may perform operations 616, 618 during runtime. At operation 616, images acquired by a camera of a video laryngoscope (e.g., the images received in operation 602 in FIG. 6) are provided into the trained ML model. The acquired images, or a portion of the acquired image (such as a lower half, lower third, lower fourth, etc. of the acquired image), may be provided as an input into the trained ML model in real time. If the training data includes cropped images, then the acquired image may be cropped accordingly prior to being provided as an input into the trained ML model. Although a cropped version of the acquired image may be displayed at a display of the video laryngoscope, regions of the acquired image not displayed may be provided as input into the trained ML model. For example, a first cropped region of the image may be displayed and a second cropped region (which may include all, some, or none of the first cropped region) may be provided as input into the trained ML model.

At operation 618, a tool detection determination is received as an output of the trained ML model. The input image (e.g., the acquired image, which may be cropped appropriately), may be received by the trained ML model and classified into one of the trained classes. The outputted tool detection from the trained ML model may then be used by the video laryngoscope (e.g., as described at operation 606, 608 in FIG. 6).

Returning to the discussion of FIG. 6, if determination 604 results in a determination that no tool is detected, the method 600 flows “NO” to operation 606. At operation 606, a display region of the acquired image is selected. The display region may have a same aspect ratio as a display of the video laryngoscope. The display region (e.g., a portion of the acquired image) may be selected based on patient anatomy detected. Additionally or alternatively, or if detection of patient anatomy is not performed, a top edge of the display region may be predetermined based on an offset or spacing from a top edge of the acquired image.

If, alternatively, determination 604 results in a determination that a tool is detected, the method 600 flows “YES” to operation 608. At operation 608, a display region of the acquired image is selected based on the detected tool(s). A display region for the acquired image may also be based on detected patient anatomy. The display region may have a same aspect ratio as a display of the video laryngoscope. The top edge of the display region may be predetermined based on an offset or spacing from a top edge of the acquired image or determined based on a distance above detected patient anatomy. The bottom edge of the display region may be based on showing at least a portion of the detected tool (e.g., showing a tool portion height or a tip of the tool).

At operation 606, the selected display region is displayed. The display region is fitted or filled to a display screen of a video laryngoscope. For example, a display region may be enlarged to fill a display, resulting in a zoom-in effect. Additionally, a display region may be shrunk or reduced to fit a display, resulting in a zoom-out effect.

Operations 602-610 may repeat as required or desired. For example, as new images are acquired by a camera of the video laryngoscope, tool detection may be performed. As one or more tools are detected, the display region may be re-selected or adjusted. The bottom edge of the display region may change distance relative to the top edge of the display region to maintain a constant view of a portion of the detected tool as the tool moves. The top edge of the display region may be constant relative to the acquired image (e.g., a constant spacing or offset from the top edge of the acquired image) and/or constant relative to patient anatomy (e.g., spaced a set distance above detected vocal cords). Thus, the display region is progressively adjusted according to movement of detected tool(s). Progressive adjustment may continue until a maximum or minimum display region is reached.

As an example, a first image may be acquired from a video feed of a camera of the video laryngoscope. A tool, such as an endotracheal tube, scope, forceps, introducer, etc., may be detected in the image (e.g., via input of all or a portion of the first image being provided as input into a trained ML model). Based on the tool detected in the first image, the video laryngoscope may automatically select a first display region of the first image that includes relevant patient anatomy (e.g., vocal cords, larynx, etc.) and a portion of the tool. A portion of the tool may be a distal tip of the tool, certain components of a tool (e.g., a tip of the endotracheal tube after a cuff), an included display height of the tool (e.g., a height of the portion of the tool shown at a display of the video laryngoscope, after selecting the display region and fitting/filling the display region to the display), etc. Similarly, a second image may be acquired from the video feed of the camera of the video laryngoscope, such as an image acquired after the first image during an intubation of a patient or training model. An amount of the tool present in the second image may be different than that of the first image (e.g., the tool moved in the airway between capture of the first image and the second image). The tool in the second image may be detected. Based on the tool detected in the second image, a second display region of the second image may be automatically selected to include the patient anatomy and the tip of the tool. The portion of the tool shown in the second display region (when displayed at the display of the video laryngoscope, such as after fitting/filling the display region to the display) may be the same as the first image. This may result in an illusion of the tool maintaining a constant distance from the camera while the patient anatomy is resized and/or moved (e.g., the patient anatomy may be enlarged or shrunk when comparing display of the first cropping image with the second cropped image). Display of the first display region and the second display region may be provided in real time.

While the automatic detection of the tool(s) described above is described as being used for zooming/cropping, additional or alternative operations may be performed based on the detection of the tools. For instance, the type of tool may also be classified as part of the detection process, which allows for determining whether a particular type of tool has been detected (e.g., endotracheal tube versus a bougie). These types of classifications may be useful in automatically generating charts to know which tools were used and when they were used during the procedure. In addition, one or more of the frames where the tool was detected may be marked as key frames for later video processing and/or review. Screenshots of those frames may be extracted and stored.

Further, in some examples, the video feed from the video laryngoscope may be transmitted or streamed to another device, such as monitor within the room, for concurrent or subsequent viewing. The transmitted or streamed video may retain the same zoom/cropping levels as the video displayed on the video laryngoscope itself. In other examples, an option may be presented to change the level of zoom or view video data outside of the zoomed region during playback of the video.

The techniques introduced above may be implemented for a variety of medical devices or devices where direct and indirect views are possible. A person of skill in the art will understand that the technology described in the context of a video laryngoscope for human patients could be adapted for use with other systems such as laryngoscopes for non-human patients or medical video imaging systems.

Those skilled in the art will recognize that the methods and systems of the present disclosure may be implemented in many manners and as such are not to be limited by the foregoing aspects and examples. In other words, functional elements being performed by a single component or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for automatic cropping by a video laryngoscope, the method comprising:
acquiring a first image from a video feed of a camera of the video laryngoscope, the first image including a first portion of a tool and patient anatomy;
detecting the tool in the first image;
based on the detected tool in the first image, automatically selecting a first display region of the first image, the first display region including the patient anatomy and a tip of the tool;
acquiring a second image from the video feed, the second image including a second portion of the tool and the patient anatomy;
detecting the tool in the second image; and
based on the detected tool in the second image, automatically selecting a second display region of the second image, the second display region including the patient anatomy and the tip of the tool.

2. The method of claim 1, wherein the patient anatomy is enlarged in the second display region relative to the first display region.

3. The method of claim 1, wherein the method further includes displaying the first display region and the second display region in real time at a display of the video laryngoscope.

4. The method of claim 3, wherein the tip of the tool in the first display region and the second display region has a same height when the first display region and the second display region are displayed, wherein the same height is a distance from an end of the tip of the tool to a bottom edge of one of the first display region or the second display region.

5. The method of claim 1, wherein the patient anatomy includes vocal cords and the tool is an endotracheal tube.

6. The method of claim 5, wherein the tip of the tool is a distal end of the endotracheal tube positioned distally from a cuff of the endotracheal tube.

7. A method for automatic cropping by a video laryngoscope, the method comprising:
acquiring an image using a camera of the video laryngoscope, the image including a portion of a tool;
providing at least a portion of the acquired image as input into a trained machine learning (ML) model;
receiving detection of the tool as output from the trained ML model;
based on the tool detection, zooming out to a portion of the acquired image including patient anatomy and a tool portion of the tool having a tool height; and
displaying the zoomed-out portion of the acquired image at a display of the video laryngoscope.

8. The method of claim 7, wherein the tool height is less than 20 mm when the zoomed-out portion of the acquired image is displayed at the display of the video laryngoscope.

9. The method of claim 7, wherein displaying the zoomed-out portion of the acquired image includes fitting the zoomed-out portion to the display.

10. The method of claim 7, wherein the aspect ratio of the zoomed-out portion and the display are the same.

11. The method of claim 7, further comprising:
detecting a progression of the tool towards the patient anatomy; and
progressively zooming in to portions of images acquired by the camera as the tool progresses towards the patient anatomy.

12. The method of claim 11, wherein the zoomed-in portions include the tool portion having the tool height.

13. A video laryngoscope comprising:
a handle portion;
a display screen coupled to the handle portion;
a camera, positioned at a distal end of the blade portion, that acquires a video feed while the video laryngoscope is powered on;
a memory; and
a processor that operates to:
acquire a first image from a video feed of a camera of the video laryngoscope,
the first image including patient anatomy;
display a first portion of the first image on the display screen;
acquire a second image from the video feed, the second image including a tool and the patient anatomy;
detect the tool in the second image; and
based on detecting the tool in the second image, display a second portion of the second image, wherein the second portion is larger than the first portion thereby providing a zoom-out effect.

14. The system of claim 13, wherein first portion is cropped from the first image, wherein the second portion is cropped from the second image, and wherein the second portion includes a tip of the tool and the patient anatomy.

15. The system of claim 13, wherein the processor further operates to:
acquire a third image from the video feed, the third image including the patient anatomy and the tool after the tool has been further distally inserted towards the patient anatomy;
detect the tool in the third image; and
based on detecting the tool in the third image, display a third portion of the third image, wherein the third portion is smaller than the second portion thereby providing a zoom-in effect as compared to the displayed second portion of the second image.
